(19) United States
(12) Patent Application Publication
(10) Pub. No.: US 2002/0061902 A1
(43) Pub. Date: May 23, 2002

(54) Treatment method

(75) Inventors: Alan G. Harris, New York, NY (US); Paul T. Medeiros, Easton, PA (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: SCHERING CORPORATION

(21) Appl. No.: 10/021,189

(22) Filed: Oct. 30, 2001

Related U.S. Application Data
(60) Provisional application No. 60/244,365, filed on Oct. 30, 2000.

(51) Int. Cl. A61K 31/496; A61K 31/473

(52) U.S. Cl. 514/290; 514/253.03

(57) ABSTRACT

Methods of treating and/or preventing a cardiovascular disease in a human suffering from an allergic and/or inflammatory condition of the skin or upper airway passages or cardiovascular disease by administrating an effective amount of an antihistamine, preferably desloratadine, alone, or in admixture with an effective amount of at least one leukotriene antagonist, preferably, montelukast, for such reducing and/or preventing are disclosed.

### BACKGROUND OF THE INVENTION

This invention relates to a method of treating and/or reducing the risk of cardiovascular disease in a human suffering from an allergic and/or inflammatory condition comprising the administration of an effective amount of an antihistamine alone, or in admixture with an effective amount of at least one leukotriene antagonist, preferably, montelukast.

Traditional risk factors implicated in the development and progression of cardiovascular diseases such as atherosclerosis and for the predisposition of unstable angina, myocardial infarction and stroke include cigarette smoking, hypertension, dyslipidemia, diabetes mellitus, sedentary lifestyle, obesity, the imbalance of the hemostatic/fibrinolytic system and a family history of premature coronary disease. However, these risk factors explain only a portion of the documented cardiovascular disease. Other factors must play a role in the etiology of the vascular events in this disease.

Recent studies have suggested immunologic mechanisms may play a role in the pathogenesis of cardiovascular disease. For example, Kovanen, P. T., et al., in Arch. Inter. Med., 1998, Vol. 158, pp.1434-1439 disclose that elevated levels of the immunoglobins IgA, IgE and IgG are associated with myocardial infarction and cardiac death in men with dyslipidemia; Criqui, M. H., et al., in The American Journal of Medicine, 1987, Vol. 82, pp. 964-968 disclose that there is a possible link between allergic disease and cardiovascular disease in men, but not in women; and Kockmaz, M.E., et al., in International Journal of Cardiology, 1991, Vol. 31, pp. 199-204, disclose that IgE serum levels were significantly higher in patients with unstable angina and acute myocardial infarction compared to patients with stable angina pectoris and normal subjects (controls).

Furthermore, it has been reported that eosinophilia is associated as an additional risk factor in death from cardiovascular diseases including ischemic heart disease and cerebrovascular disease. Hospes, et al., Am. J. Epidemiol., Vol. 150, (No. 5), pp. 482-491, (1999).

Ciprandi, G., et al., Clinical and Experimental Allergy, (1997) Vol. 27 (No.10):1 175-1183, disclose that cetirizine and loratadine given over a two week period to patients suffering from rhinitis due to pollen allergy reduced the rhinitis symptoms and reduced eosinophil counts and ICAM-1 expression on nasal epithelial cells.

Corssmit, E. P., et al., Cardiology, (1999), Vol. 91 (No.4): 272-276 disclose that a cortiocosteroid, e.g., prednisone, is the drug of choice for treating diseases characterized by sustained eosinophilia and/or caused by infiltration of eosinophils, such as Loffler's endo-myocardititis and idiopathic hypereosinophilic syndrome. If prednisone fails, Corssmit recomends that myelosuppressive drugs, such as hydroxyurea, vincristine or interferon-alpha, be administered. Such drugs are cytotoxic or have undesireable side effects.

Danesh, J., et al., British Medical Journal, 2000, Vol. 321, 199-204 suggest that low grade systemic inflammation unrelated to chronic infection may be an integral part of the atherosclerotic process, and thus likely to be involved in progression to coronary heart disease.

The products of the 5-lipoxygenase pathway of arachidonic acid metabolism, particularly the leukotrienes (e.g., cysteinyl leukotrienes, LTC4, LTD4, LTE4) are released from various cells, including mast cells and esosinophilis, and can mediate bronchoconstriction, mucous secretion, airway mucosal edema, chemotaxis and mobilization of cells into the airway in the inflammatory process of asthma. Leukotriene antagonists are useful in treating and preventing asthma by inhibiting the physiologic action of the leukotrienes. It would be highly desirable to enhance the efficacy of such leukotriene antagonists to improve their overall efficacy.

Thus, immunologic responses to allergic and/or inflammatory conditions have been implicated in increased risk of cardiovascular diseases, and in some cases, even death. Accordingly, there is a need for a safer, clinically effective therapy to reduce or prevent a cardiovascular disease in patients at risk.

### SUMMARY OF THE INVENTION

We have discovered a safe and effective therapy for a human at risk of or suffering from a cardiovascular disease by administering an effective amount of an antihistamine for a time sufficient to reduce the risk or prevent the occurrence of a cardiovascular disease.

Thus, the present invention provides a method of treating and/or preventing a cardiovascular disease in a human suffering from an allergic and/or inflammatory condition of the skin or upper airway passages which comprises administering to such human in need of such reducing and/or preventing an effective amount of an antihistamine, or a pharmaceutically acceptable salt thereof.

The present invention also provides a method of treating and/or preventing cardiovascular diseases in a human in need of or such treating and/or preventing which comprises administering to such human an effective amount of an antihistamine, or a pharmaceutically acceptable salt thereof.

The present invention also provides a method of treating and/or preventing a cardiovascular disease in a human suffering from seasonal or perennial allergic rhinitis which comprises administering to such human an effective amount of an antihistamine, or a pharmaceutically acceptable salt thereof.

The present invention also provides a method of treating and/or preventing cardiovascular diseases associated with an allergic and/or inflammatory condition of the skin or upper airway passages which comprises administering to such human in need of or such treating and/or preventing an effective amount of an antihistamine, or a pharmaceutically acceptable salt thereof.

The present invention also provides a method of treating and/or preventing a cardiovascular disease in a human suffering from atopic dermatitis or urticaria which comprises administering to such human an effective amount of an antihistamine, or a pharmaceutically acceptable salt thereof in admixture with at least one leukotriene antagonist, or a pharmaceutically acceptable salt thereof.

The present invention also provides a method of treating and/or preventing cardiovascular diseases in association with an allergic and/or inflammatory condition of the skin or upper airway passages which comprises administering to a human in need of or such treating and/or preventing an effective amount of an desloratadine, or a pharmaceutically acceptable salt thereof, in admixture with an effective amount of montelukast, or a pharmaceutically acceptable salt thereof.

### DETAILED DESCRIPTION OF INVENTION

We have found that common immunological cells/mediators or messengers which are responsible for disease expression in allergy also subsequently increases the risk and/or severity of cardiovascular disease. These immunological cells include mast cells, eosinophils and neutrophils, while immunological mediators or messengers include cytokines (e.g., IL-4, IL-13, GM-CSF, TNFa, IL-6), chemokines, adhesion molecules, and mast cells mediators (leukotrienes, histamine). Eosinophils and mast cells are present in the heart and major blood vessels. We have recognized that cardiac mast cell activation can occur, and that down-regulation of systemic inflammation by anti-inflammatory therapies of the present invention lowers the risk and/or severity of atherosclerosis and cardiovascular disease.

Mast cells are present in cardiac muscle, more specifically in the blood vessel wall (intima and adventitia), as well in human atherosclerotic blood vessel wall, preferentially at the important ‘shoulder’ region of the plaque. Activated mast cells increase in coronary arterial atheroma plaque. Cardiac mast cells contain histamine, tryptase, and chymase. Chymase can convert Angiotensin I to Angiotensin II, which may increase cardiovascular risk by raising blood pressure. Angiotensin II may also play a role in the proliferation of smooth muscle cells that helps form atherosclerotic plaques. Chymase may cleave bound LDL, thereby freeing it to be incorporated into atheroma by macrophages. Mast cell numbers are increased in a heart that has myocardial ischemia. By lowering immunological cells/mediators in accordance with the methods of the present invention, we lower the risk and/or severity of cardiovascular disease.

An eosinophil is a type of white blood cell which normally represents about 8% of the total white blood cell population in the circulating blood. Eosinophilia is the formation and the accumulation of eosinophils above the normal level of about 350 copies per μL of peripheral blood. The development of eosinophilia has features of an immune response and occurs in diseases, including seasonal and perennial allergic rhinitis, asthma, urticaria, eczema, atopic dermatitis, parsite infections, drug reactions and connective tissue disease, such as rheumatoid arthritis and scleroderma. Infiltration of the airways by eosinophils is an especially important factor in the development of airway inflammation that contributes to the pathophysiology of bronchial asthma and allergic rhinitis.

The phrase “an allergic and/or inflammatory condition of the skin or airway passages” as used herein means those allergic and/or inflammatory conditions and symptoms found on the skin and in the airway passages from the nose to the lungs. Typical allergic and/or inflammatory conditions of the skin and upper and lower airway passages include seasonal and perennial allergic rhinitis, non-allergic rhinitis, asthma including allergic and non-allergic asthma, sinusitis, colds, dermatitis, especially allergic and atopic dermatitis and urticaria. Inhibition of eosinophil infiltration and/or function may be implicated in the reduction of airway inflammation and thus alleviate development of bronchial asthma and allergic rhinitis.

Typically suitable eosinophilia-related and immunoglobin-related allergic and/or inflammatory conditions of the skin or the upper and lower airway passages include, but are not limited to, allergic asthma, seasonal allergic rhinitis, perennial allergic rhinitis, atopic dermatitis, chronic obstructive lung disease.

The term “cardiovascular disease” means diseases related to the heart and the blood vessels or the circulation, such as atherosclerosis, ischemic heart disease or cerebrovascular disease such as coronary artery disease including angina pectoris and myocardial infarction, stroke, vascular heart disease and peripheral vascular disorders such as peripheral arterial disease and occlusive arterial diseases.

We have found that administering therapeutically effective amounts of an antihistamine, preferably desloratadine is useful in treating and/or preventing cardiovascular disease in patients, especially those patients suffering from an allergic and/or inflammatory condition of the skin or upper airway passages. In a preferred embodiment of the present invention, an antihistamine, preferably desloratadine, is administered to those patients, such as type 2 diabetic patients or patients with asthma or asthma and seasonal allergic rhinitis afflicted with minimal persistent allergic inflammation to prevent or lower the risk of developing cardiovascular disease. In another embodiment, desloratadine is administered to those patients, such as type 2 diabetic patients or patients afflicted with asthma or asthma in addition to seasonal allergic rhinitis, afflicted atherosclerotic disease to prevent or lower the risk of developing cardiovascular disease.

In another preferred embodiment, an anti-histamine, such as but not limited to, desloratadine, is administered in admixture with at least one, preferrably one, leukotriene antagonist. In another preferred embodiment desloratadine is administered in admixture with montelukast.

The term “patients in need of such treating and/or preventing” as used herein means those patients at risk of cardiovascular disease as identified by traditional coronary risk factors enumerated above, as well as those having an allergic and/or inflammatory condition of the skin or airway passages, elevated serum levels of eosinophils and/or immunoglobulin levels, e.g., IgA, IgE, IgG and IgM compared to those found in normal subjects. Immunoglubin and eosinophil serum levels may be measured by standard commercially available quantitative immunoturbidimetry, e.g., an automated clinical chemistry analyzer (KoneSpecific R, Kone Instruments, Espoo, Finland). IgE serum levels may also be measured using an automated microparticle enzyme immunoassay such as IMx available from Abbott Diagnostics, U.S.A. and serum IgG levels may be also assessed by nephelometry (Behring, Germany).

The antihistamines useful in the present invention include descarboethoxyloratidine, cetirizine, fexofenadine, ebastine, astemizole, norastemizole, epinastine, efletirizine or a pharmaceutically acceptable salt thereof. The use of desloratadine is most preferred.

Cetirizine is disclosed in U.S. Pat. No. 4,525,358. Preferably the pharmaceutically acceptable salt is the hydrochloride, also known as cetirizine hydrochloride. The amount of cetirizine which can be employed in a unit dosage form of the present composition can range from about 2.5 to 20 mg, also from about 5 to about 10 milligrams, preferably about 10 milligrams.

Fexofenadine (MDL 16,455A) is disclosed in U.S. Pat. No. 4,254,129. Preferably the pharmaceutically acceptable salt is the hydrochloride, also known as fexofenadine hydrochloride. The amount of fexofenadine which can be employed in a unit dosage form of the present composition can range from about 40 to 200 mg, also from about 60 to about 180 milligrams, also about 120 milligrams.

Ebastine is described in EP 134124. The amount of ebastine which can be employed in a unit dosage form can range from about 5 to about 20 mg, preferably about 10 mg.

Astemizole is described in U.S. Patent No. 4,219,559. The amount of astemizole which can be employed in a unit dosage form can range from about 5 to about 20 mg, preferably about 10 mg.

Norastemizole is an antihistamine, whose technical name is 1-((4-fluorophenyl)methyl)-N-4-piperidinyl-1H-benzimidazol-2-amine. CAS Reg. No. 75970-99-9. The compound is an active metabolite of astemizole. The amount of norastemizole which can be employed in a unit dosage form can range from about 5 to about 40 mg, also from about 10 to about 20 mg.

Epinastine is described in DE 3008944 or Jpn. J. Clin. Pharmocol. Ther., 1991, 22, page 617. The amount of epinastine which can be employed in a unit dosage form can range from about 1 to about 20 mg, preferably about 2 to about 18 mg.

Efletirizine (UCB-28754) is an antihistamine, whose technical name is [2-[4-[Bis(p-fluorophenyl)methyl]-1-piperazinyl]ethoxy]acetic acid. CASReg. No.140756-35-7. The amount of efletirizine which can be employed in a unit dosage form can range from about 4 to about 60 mg.

Leukotriene D4 antagonist found useful in the present invention include, but are not limited to:

a) montelukast;

b) 1-(((R)-(3-(2-(6,7-difluoro-2-quinolinyl)ethenyl)phenyl)-3-(2-(2-hydroxy-2-propyl)phenyl)thio)methylcyclopropaneacetic acid;

c) 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneacetic acid;

d) praniukast;

e) zafirlukast; or

f) [2-[[2-(4-tert-butyl-2-thiazolyl)-5-benzofuranyl]oxymethyl]-phenyl]acetic acid; or a pharmaceutically acceptable salt thereof.

The amount of leukotriene antagonist which can be employed in a unit dosage form can range from about 5 to about 500 milligrams, also from about 50 to about 300 milligrams, also from about 100 to about 200 milligrams.

Montelukast is a leukotriene D4 antagonist capable of antagonizing the receptors for the cysteinyl leukotrienes. The technical name of montelukast is [R-(E)]-1-[[[1-[3-[2-(7-chloro-2-quinolinyl)ethenyl]phenyl]-3-[2-(1-hydroxy-1-methylethyl)phenyl]propyl]thio]methyl]-cyclopropaneacetic acid. This compound is described in EP 480,717. A preferred pharmaceutically acceptable salt of montelukast is the monosodium salt, also known as montelukast sodium. The amount of montelukast which can be employed in a unit dosage form of the present invention can range from about 1 to 100 milligrams, also from about 5 to about 20 milligrams, preferably about 10 milligrams.

The compound 1-(((R)-(3-(2-(6,7-d ifluoro-2-quinolinyl)ethenyl)phenyl)-3-(2-(2-hydroxy-2-propyl)phenyl)thio)methylcyclopropaneacetic acid is a leukotriene antagonist described in WO 97/28797 and U.S. Pat. No. 5,270,324. A pharmaceutically acceptable salt of this compound is the sodium salt, also known as sodium 1-(((R)-(3-(2-(6,7-difluoro-2-quinolinyl)ethenyl)phenyl)-3-(2-(2-hydroxy-2-propyl) phenyl)thio)-methylcyclopropaneacetate.

The compound 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)-thio)methyl)cyclopropaneacetic acid is a leukotriene antagonist described in WO 97/28797 and U.S. Pat. No. 5,472,964. A pharmaceutically acceptable salt of this compound is the sodium salt, also known as sodium 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)-thio)methyl)cyclopropaneacetate.

Pranlukast is a leukotriene antagonist described in WO 97/28797 and EP 173,516. The technical name for this compound is N-[4-oxo-2-(1H-tetrazol-5-yl)-4H-1-benzopyran-8-yl]-p-(4-phenylbutoxy)benzamide. The amount of pranlukast which can be employed in a unit dosage form can range from about 100 to about 700 mg, preferably from about 112 to about 675 mg; also from about 225 mg to about 450 mg; also from about 225 to about 300 mg.

Zafirlukast is a leukotriene antagonist described in WO 97/28797 and EP 199,543. The technical name for this compound is cyclopentyl-3-[2-methoxy-4-[(o-tolylsulfonyl)carbamoyl]benzyl]-1-methylindole-5-carbamate.

The compound [2-[[2-(4-tert-butyl-2-thiazolyl)-5 benzofuranyl]oxymethyl]phenyl]acetic acid is a leukotriene antagonist and/or inhibitor whose method for preparation is described in U.S. Pat. No. 5,296,495 and Japanese Patent JP 08325265 A. An alternative name for this compound is 2-[[[2-[4-(1,1-dimethylethyl)-2-thiazolyl]-5-benzofuranyl]oxy]methyl]-benzeneacetic acid. The code number for this compound is FK011 or FR150011. The compound has a molecular formula of C₂₄H₂₃NO₄S and molecular weight of 421.52.

The pharmaceutical compositions of the present invention can be administered depending upon the patient's age, sex, weight and severity of the condition being treated. Generally, the human oral dosage form containing descarboethoxyloratidine, cetirizine, fexofenadine, ebastine, astemizole, norastemizole, epinastine, efletirizine or a pharmaceutically acceptable salt thereof and the leukotriene antagonist can be administered 1 or 2 times per day.

The term “in admixture with” as used herein means that the antihistamines may be administered contemporaneously or sequentially with the leukotriene antagonist as separate pharmaceutical compositions or together in one composition.

Preferably the pharmaceutical composition is designed for oral adminstration. Preferably the leukotriene antagonist is montelukast and the pharmaceutically acceptable salt of monoleukast is montelukast sodium. Also preferred is that the pharmaceutically acceptable salt of monoleukast is about 10 milligrams (mg). Most preferably the antihistamine is descarboethoxyloratidine. Preferably, a pharmaceutically acceptable salt of cetirizine or fexofenadine is the hydrochloride salt. Also preferred is that descarboethoxyloratidine or cetirizine is about 2.5 to about 20 mg, more preferably about 5, 7.5 or 10 mg. Preferably, fexofenadine is from about 60 to 180 mg. More preferably, the pharmaceutically acceptable salt of monteleukast is about 10 mg and descarboethoxyloratidine is about 5 or 7.5 mg.

Preferably the leukotriene antagonist is montelukast and the pharmaceutically acceptable salt of monoleukast is montelukast sodium. Also preferred is that the pharmaceutically acceptable salt of monoleukast is about 10 milligrams (mg). Most preferably the antihistamine is descarboethoxyloratidine. Preferably, a pharmaceutically acceptable salt of cetirizine or fexofenadine is the hydrochloride salt. Also preferred is that descarboethoxyloratidine or cetirizine is about 2.5 to about 20 mg, more preferably about 5, 7.5 or 10 mg. Preferably, fexofenadine is from about 60 to about 180 mg. More preferably, the pharmaceutically acceptable salt of monteleukast is about 10 mg and descarboethoxyloratidine is about 5 or 7.5 mg.

The present invention also contemplates use of an antihistamine in combination with one of more of the therapies useful for lowering serum cholesterol levels. Such therapies include Hormone Replacement therapies, e.g., Premarin, raloxifene hydrochloride, available from Eli Lilly under the EVISTA tradename, as well as hypocholesterolemic agents such as ezetimibe disclosed in U.S. Pat. No. 5,767,115, and cholesterol biosynthesis inhibitors.

The term “cholesterol biosynthesis inhibitors” include 3-hydroxy-3-methylglutaryl coenzyme A (HMG CoA) reductase inhibitors, such as lovastatin, pravastatin, fluvastatin, itavastatin, simvastatin, ZD-4522 (available from AstraZeneca), and CI-981, as well as HMG CoA synthesis inhibitors, including for example, squalestatin 1, and squalene synthesis inhibitors, for example, NB-598 and other cholesterol biosynthesis inhibitors such as DMP-565. The preferred HMG CoA reductase inhibitors are lovastatin, itavastatin, simvastatin, and ZD-4522.

U.S. Pat. No. 4,659,716 discloses methods of making desloratadine, pharmaceutical compositions containing it and methods of using desloratadine and pharmaceutical compositions containing it to treat allergic reaction in mammals.

U.S. Pat. No. 5,595,997 discloses pharmaceutical compositions containing desloratadine and methods of using desloratadine for treating and preventing various disease states, e.g., allergic rhinitis and other disorders.

Desloratadine is available from Schering-Plough Corporation, Kenilworth, N.J.

The amount of desloratadine effective for use in the present invention will vary with the age, sex, body weight, severity of the allergic and inflammatory condition and the response of the patient. Typically, the amount of desloratadine effective for treating or preventing such allergic and inflammatory conditions is in the range of about 2.5 mg/day to about 45 mg/day, preferably about 2.5 mg/day to about 20 mg/day, or about 5.0 mg/day to about 15 mg/day, or about 5.0 mg/day to about 10 mg/day, more preferably about 5.0 mg/day to about 7.5 mg/day, and most preferably about 5.0 mg/day in single or divided doses, or a single dose of 5.0 mg/day.

Treatment should be continued until there is improvement in the patient's condition. Lower immonuglobin and /or eosinophil levels (compared to baseline levels) in the patients treated in accordance with the present invention indicates improvement in the patient's condition and risk for cardiovascular disease.

Improvement in the patients at risk may also be ascertained upon review of a complete physical and serological examination of the patient by an attending clinician.

Desloratadine can be employed in pharmaceutical compositions. Such pharmaceutical compositions may be formulated by combining desloratadine or an equivalent amount of a pharmaceutically acceptable salt thereof with a suitable, inert, pharmaceutically acceptable carrier or diluent that may be either solid or liquid.

Desloratadine may be converted into the pharmaceutically acceptable acid addition salts by admixing it with an equivalent amount of a pharmaceutically acceptable acid. Typically suitable pharmaceutically acceptable acids include mineral acids, e.g., HNO₃, H₂SO₄, H₃PO₄, HCI, HBr, HI, organic acids, including, but not limited to, aliphatic, aromatic, carboxylic, and sulfonic classes of organic acids, examples of which are formic, acetic, trifluoroacetic, propionic, lactic, maleic, succinic, tartaric, glucuronic, glycolic, furoic, glutamic, benzoic, anthranilic, salicylic, phenylacetic, mandelic, tothenic, stearic, sulfanilic, algenic, galacturonic and citric acids as well as alkyl or arylsulfonic acids, such as p-toluenesulfonic acid, 2-naphthalenesulfonic acid, benzenesulfonic or methanesulfonic acid. The preferred pharmaceutically acceptable salts are trifluoroacetate, tosylate, mesylate, citrate and chloride. Desloratadine is more stable as the free base than as an acid addition salt and the use of the desloratadine free base in pharmaceutical compositions of the present invention is more preferred.

Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection. Solid form preparations may be converted into liquid preparations shortly before use for either oral or administration. Parenteral forms to be injected intravenously, intramuscularly or subcutaneously are usually in the form of sterile solutions and may contain tonicity agents (salts or glucose), and buffers. Opacifiers may be included in oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in a unit dosage form. In such form, the preparation is subdivided into suitably sized unit doses containing appropriate quantities of the active component, e.g., an effective amount to achieve the desired purpose.

The pharmaceutical compositions of desloratadine can be adapted for any mode of administration e.g., for oral, parenteral, e.g., subcutaneous (“SC”), intramuscular (“IM”), intravenous (“IV)” and intraperitoneal (“IP”), topical or vaginal administration or by inhalation (orally or intranasally). Preferably desloratadine is administered orally.

### EXAMPLE

Desloratadine attenuation of the eosinophil chemotaxis, adhesion and superoxide generation. Eosinophils were isolated and purified (>99% pure and >98% viable) from the blood of patients with allergic rhinitis or allergic asthma; a 2-week washout preceded blood collection. Chemotaxis in response to 1 μM platelet activating factor (“PAF”); blind well-chamber technique; adhesion of ⁵¹Cr-labeled eosinophils to human umbilical endothelial cells (HUVECs) in response to 10 mg/mL TNFA; and superoxide generation (detected by reduction of cytochrome C) both spontanenous and stimulated by 10 ng/mL phorbol myristate acetate (PMA) were measured in the presence of desloratadine (0.1 to 10 μM).

Desloratadine attenuated PAF-induced eosinophil chemotaxis in a dose-dependent manner. The maximum attenuation was 36%±8% at 10 μM (P<0.02). TNFα-induced eosinophil adhesion to HUVECs was likewise attenuated by desloratadine in a dose-dependent manner, with a maximum attenuation of 27%±5% at 10 μM (P<0.02). Spontaneous superoxide generation was attenuated by desloratadine in a concentration-dependent manner, with PMA-stimulated superoxide generation also being attenuated by 10 μM desloratadine. There were no differences in the effects of desloratadine on chemotaxis, adhesion, or superoxide generation in eosinophils isolated from the blood of patents with allergic rhinitis or from those with allergic asthma.

We believe that the modulation of eosinophil recruitment and function by desloratadine contribute to its anti-inflammatory and anti-allergenic properties and provide the treating and/or preventing eosinophilia-related cardiovascular diseases.

While the invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

We claim:

1. A method of treating and/or preventing a cardiovascular disease in a human suffering from an allergic and/or inflammatory condition of the skin or upper airway passages which comprises administering to such human in need of such treating and/or preventing an effective amount of an antihistamine, or a pharmaceutically acceptable salt thereof.

2. The method of claim 1, wherein the antihistamine is one of desloratidine, cetirizine, fexofenadine, ebastine, astemizole, norastemizole, epinastine, efletirizine, or a pharmaceutically acceptable salt thereof.

3. The method of claim 1, wherein the antihistamine is desloratadine and the effective amount of desloratadine is about 2.5 mg/day to about 45 mg/day.

4. The method of claim 3, wherein the antihistamine is desloratadine and the effective amount of desloratadine is about 5 mg/day to about 15 mg/day.

5. The method of claim 4, wherein the antihistamine is desloratadine and the effective amount of desloratadine is about 5 mg/day to about 10 mg/day.

6. The method of claim 5, wherein the antihistamine is desloratadine and the effective amount of desloratadine is about 5 mg/day.

7. The method of claim 1, wherein the allergic and/or inflammatory condition of the skin or airway passages is seasonal allergic rhinitis, perennial allergic rhinitis, atopic dermatitis, urticaria or allergic asthma.

8. The method of claim 1, wherein an effective amount of at least one leukotriene antagonist, or a pharmaceutically acceptable salt thereof, is administered in admixture with the antihistamine.

9. The method of claim 1, wherein an effective amount of montelukast, or a pharmaceutically acceptable salt thereof, is administered in admixture with the antihistamine.

10. A method of treating and/or preventing a cardiovascular disease in a human in need of such treating and/or preventing which comprises administering to such a human an effective amount of an antihistamine, or a pharmaceutically acceptable salt thereof.

11. The method of claim 10, wherein the antihistamine is one of desloratidine, cetirizine, fexofenadine, ebastine, astemizole, norastemizole, epinastine, efletirizine, or a pharmaceutically acceptable salt thereof.

12. The method of claim 10, wherein the antihistamine is desloratadine and the effective amount of desloratadine is in the range of about 2.5 mg/day to about 45 mg/day.

13. The method of claim 12, wherein the antihistamine is desloratadine and the effective amount of desloratadine is about 5 mg/day to about 15 mg/day.

14. The method of claim 13, wherein the antihistamine is desloratadine and the effective amount of desloratadine is about 5 mg/day to about 10 mg/day.

15. The method of claim 14, wherein the antihistamine is desloratadine and the effective amount of desloratadine is about 5 mg/day.

16. The method of claim 10, wherein an effective amount of at least one leukotriene antagonist, or a pharmaceutically acceptable salt thereof, is administered in admixture with the antihistamine.

17. The method of claim 10, wherein an effective amount of montelukast, or a pharmaceutically acceptable salt thereof, is administered in admixture with the antihistamine.

18. A method of treating and/or preventing a cardiovascular disease in a human suffering from seasonal or perennial allergic rhinitis which comprises administering to such human in need of such treating and/or preventing an effective amount of an antihistamine, or a pharmaceutically acceptable salt thereof.

19. The method of claim 18, wherein the antihistamine is one of desloratidine, cetirizine, fexofenadine, ebastine, astemizole, norastemizole, epinastine, efletirizine, or a pharmaceutically acceptable salt thereof.

20. The method of claim 18, wherein the antihistamine is desloratadine and the effective amount of desloratadine is about 2.5 mg/day to about 45 mg/day.

21. The method of claim 20, wherein the antihistamine is desloratadine and the effective amount of desloratadine is about 5 mg/day to about 15 mg/day.

22. The method of claim 21, wherein the antihistamine is desloratadine and the effective amount of desloratadine is about 5 mg/day to about 10 mg/day.

23. The method of claim 22, wherein the antihistamine is desloratadine and the effective amount of desloratadine is about 5 mg/day.

24. The method of claim 18, wherein the antihistamine is desloratadine and the human is suffering from seasonal allergic rhinitis.

25. The method of claim 18, wherein the antihistamine is desloratadine and the human is suffering from perennial allergic rhinitis.

26. The method of claim 18, wherein an effective amount of at least one leukotriene antagonist, or a pharmaceutically acceptable salt thereof, is administered in admixture with the antihistamine.

27. The method of claim 18, wherein an effective amount of montelukast, or a pharmaceutically acceptable salt thereof, is administered in admixture with the antihistamine.

28. A method of treating and/or preventing a cardiovascular disease in a human suffering from atopic dermatitis or urticaria which comprises administering to such human in need of such treating and/or preventing an effective amount of an antihistamine, or a pharmaceutically acceptable salt thereof.

29. The method of claim 28, wherein the antihistamine is one of descarboethoxyloratidine, cetirizine, fexofenadine, ebastine, astemizole, norastemizole, epinastine, efletirizine, or a pharmaceutically acceptable salt thereof.

30. The method of claim 28, wherein the antihistamine is desloratadine and the effective amount of desloratadine is about 2.5 mg/day to about 45 mg/day.

31. The method of claim 30, wherein the antihistamine is desloratadine and the effective amount of desloratadine is about 5 mg/day to about 15 mg/day.

32. The method of claim 31, wherein the antihistamine is desloratadine and the effective amount of desloratadine is about 5 mg/day to about 10 mg/day.

33. The method of claim 32, wherein the antihistamine is desloratadine and the effective amount of desloratadine is about 5 mg/day.

34. The method of claim 28, wherein the patient is suffering from atopic dermatitis.

35. The method of claim 28, wherein the patient is suffering from urticaria.

36. The method of claim 28, wherein an effective amount of at least one leukotriene antagonist, or a pharmaceutically acceptable salt thereof, is administered in admixture with the antihistamine.

37. The method of claim 28, wherein an effective amount of montelukast, or a pharmaceutically acceptable salt thereof, is administered in admixture with the antihistamine.

38. A method of treating and/or preventing cardiovascular diseases associated with an allergic and/or inflammatory condition of the skin or upper airway passages in a human which comprises administering to the human in need of or such treating and/or preventing an effective amount of an antihistamine, or a pharmaceutically acceptable salt thereof in admixture with at least one leukotriene antagonist, or a pharmaceutically acceptable salt there of.

39. The method of claim 38, wherein the antihistamine is one of descarboethoxyloratidine, cetirizine, fexofenadine, ebastine, astemizole, norastemizole, epinastine, efletirizine, or a pharmaceutically acceptable salt thereof.

40. The method of claim 38, wherein the antihistamine is desloratadine and the effective amount of desloratadine is about 2.5 mg/day to about 45 mg/day.

41. The method of claim 40, wherein the antihistamine is desloratadine and the effective amount of desloratadine is about 5 mg/day to about 15 mg/day.

42. The method of claim 42 wherein the antihistamine is desloratadine and the effective amount of desloratadine is about 5 mg/day to about 10 mg/day.

43. The method of claim 42, wherein the antihistamine is desloratadine and the effective amount of desloratadine is about 5 mg/day.

44. The method of claim 38, wherein the allergic and/or inflammatory condition of the skin or airway passages is seasonal allergic rhinitis, perenial allergic rhinitis, atopic dermatitis, urticaria or allergic asthma.

45. The method of claim 38, wherein an effective amount of montelukast, or a pharmaceutically acceptable salt thereof, is administered in admixture with the antihistamine.

46. A method of treating and/or preventing cardiovascular diseases in a human associated with an allergic and/or inflammatory condition of the skin or upper airway passages which comprises administering to a human in need of or such treating and/or preventing an effective amount of an desloratadine, or a pharmaceutically acceptable salt thereof in admixture with an effective amount of montelukast, or a pharmaceutically acceptable salt thereof.

47. The method of claim 46, wherein the effective amount of desloratadine is about 5 mg/day to about 10 mg/day.

48. The method of claim 47, wherein the effective amount of desloratadine is about 5 mg/day.

49. The method of claim 46, wherein the effective amount of montelukast is about 5 mg/day to about 20 mg/day.

50. The method of claim 48, wherein the effective amount of montelukast is about 10 mg/day.

51. The method of claim 46, wherein the allergic and/or inflammatory condition of the skin or airway passages is seasonal allergic rhinitis, perennial allergic rhinitis, atopic dermatitis, urticaria or allergic asthma.
